(19) United States
(12) Patent Application Publication
(10) Pub. No.: US 2021/0366598 A1
(43) Pub. Date: Nov. 25, 2021

(54) System, Method, and Computer Program Product for Determining a Care Provider Based on a Care Request

(71) Applicant: Orchestrall Healthcare Limited, North Point (HK)

(72) Inventors: Man-Cheung Ian Hau, Villanova, PA (US); Daniel Allen Hirschfeld, Timonium, MD (US)

(21) Appl. No.: 17/328,087

(22) Filed: May 24, 2021

Related U.S. Application Data
(60) Provisional application No. 63/028,829, filed on May 22, 2020.

(51) Int. Cl. G16H 40/20; G06Q 40/08

(52) U.S. Cl. CPC G16H 40/20; G06Q 40/08

(57) ABSTRACT

Provided are computer-implemented methods for determining a care provider based on a care request which may include receiving data associated with a care request, the data associated with the care request transmitted by a device associated with a care recipient; determining one or more values corresponding to one or more parameters of the care request; determining one or more weighted scores corresponding to one or more care providers based on the one or more values corresponding to the one or more parameters of the care request; and selecting a care provider from among the one or more care providers based on the one or more weighted scores. In some non-limiting embodiments or aspects, methods may include generating a care request for the care provider selected from among the one or more care providers. Systems and computer program products are also provided.

### CROSS REFERENCE TO RELATED APPLICATIONS

This application claims the benefit of U.S. Provisional Application No. 63/028,829 filed May 22, 2020, incorporated herein by reference in its entirety.

### BACKGROUND

### Technical Field

This disclosure relates generally to providing response to care requests and, in some non-limiting embodiments or aspects, to systems, methods, and computer program products for determining a care provider based on a care request.

### Technical Considerations

Care providers (e.g., a clinician such as a doctor, a physical therapist, and/or the like, a relative, a friend, and/or the like) caring for a care recipient (e.g., a patient, a customer, and/or the like) may receive a request from the care recipient or family caregiver for services (e.g., a request to schedule an appointment). The individual caring for the care recipient may then determine when to schedule the request for services. However, the individual may not be able to accommodate the request from the care recipient for services as quickly and/or may not be as well suited to accommodate the request as another individual. For example, the individual may be trained to generally provide the services but may not be an individual who specializes in the services. Additionally, the identity of another individual may not be known to the individual caring for the care recipient. As a result, the care recipient may wait until the individual caring for the care recipient is available, resulting in a potential delay in the care recipient receiving services from an individual who is otherwise capable of providing the services requested.

### SUMMARY

Accordingly, disclosed are systems, methods, and computer program products for determining a care provider based on a care request.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the present disclosure. As used in the specification and the claims, the singular form of “a,” “an,” and “the” include plural referents unless the context clearly dictates otherwise.

### BRIEF DESCRIPTION OF THE DRAWINGS

FIG. 1 is a diagram of a non-limiting aspect or embodiment of a system for determining a care provider based on a care request;

FIG. 2 is a diagram of a non-limiting aspect or embodiment of components of one or more devices and/or one or more systems of FIG. 1;

FIG. 3 is a flowchart of a non-limiting aspect or embodiment of a process for determining a care provider based on a care request; and

[symbol]

### DETAILED DESCRIPTION

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the disclosure as it is oriented in the drawing figures. However, it is to be understood that the disclosure may assume various alternative variations and step sequences, except where expressly specified to the contrary. It is also to be understood that the specific devices and processes illustrated in the attached drawings, and described in the following specification, are simply exemplary embodiments or aspects of the disclosure. Hence, specific dimensions and other physical characteristics related to the embodiments or aspects of the embodiments disclosed herein are not to be considered as limiting unless otherwise indicated.

No aspect, component, element, structure, act, step, function, instruction, and/or the like used herein should be construed as critical or essential unless explicitly described as such. In addition, as used herein, the articles “a” and “an” are intended to include one or more items and may be used interchangeably with “one or more” and “at least one.” Furthermore, as used herein, the term “set” is intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or send (e.g., transmit) information to the other unit. This may refer to a direct or indirect connection that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if one or more intermediary units (e.g., a third unit located between the first unit and the second unit) processes information received from the first unit and transmits the processed information to the second unit. In some non-limiting embodiments or aspects, a message may refer to a network packet (e.g., a data packet and/or the like) that includes data.

As used herein, the term “server” may refer to one or more devices, such as processors, storage devices, and/or similar components that communicate with client devices and/or other devices over a network, such as the Internet or private networks and, in some examples, facilitate communication among other servers and/or client devices.

As used herein, the term “system” may refer to one or more devices or combinations of devices such as, but not limited to, processors, servers, client devices, software applications, and/or other like components. In addition, reference to “a server” or “a processor,” as used herein, may refer to a previously-recited server and/or processor that is recited as performing a previous step or function, a different server and/or processor, and/or a combination of servers and/or processors. For example, as used in the specification and the claims, a first server and/or a first processor that is recited as performing a first step or function may refer to the same or different server and/or a processor recited as performing a second step or function.

Provided are improved systems, methods, and computer program products for determining a care provider based on a care request. In some non-limiting embodiments or aspects, systems, methods, and computer program products may include receiving data associated with a care request, the data associated with the care request transmitted by a device associated with a care recipient; determining one or more values corresponding to one or more parameters of the care request; determining one or more weighted scores corresponding to one or more care providers based on the one or more values corresponding to the one or more parameters of the care request; and selecting a care provider from among the one or more care providers based on the one or more weighted scores.

By virtue of implementation of the systems, methods, and computer program products described herein, an individual that is able to care for a care recipient based on a request from the care recipient for services may be selected where the individual is able to accommodate the request from the care recipient for services quicker than an individual selected by the care recipient. As a result, the care recipient may not need to wait until the individual caring for the care recipient is available, resulting in a potential reduction in delay in the care recipient receiving services from an individual that is otherwise capable of providing the services requested. Further, systems, methods, and computer program products described herein may result in more accurate selection of individuals able to care for a care recipient than may be selected by the care recipient, leading to a reduction in the consumption of network resources.

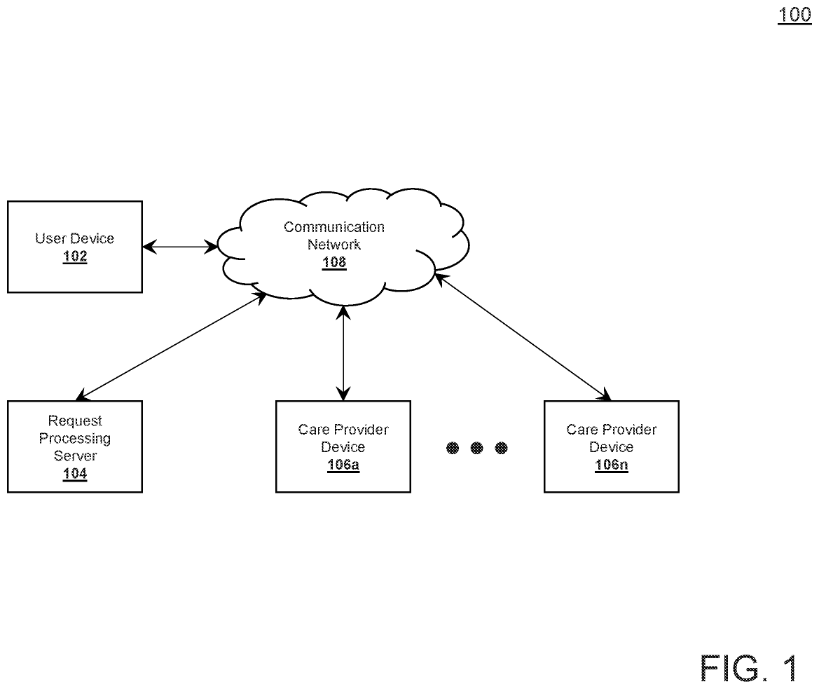

Referring now to FIG. 1, FIG. 1 is a diagram of an example environment 100 in which devices, systems, methods, and/or products described herein may be implemented. As shown in FIG. 1, environment 100 includes user device 102, request processing server 104, and/or care provider devices 106a-106n (referred to collectively as “care provider devices 106” and individually as “care provider device 106”). User device 102, request processing server 104, and/or care provider devices 106 may interconnect (e.g., establish a connection to communicate, and/or the like) via wired connections, wireless connections, or a combination of wired and wireless connections.

User device 102 may include a device configured to be in communication with request processing server 104 and/or care provider devices 106 via communication network 108. For example, user device 102 may include a mobile device (e.g., a smartphone, a tablet, and/or the like), a laptop computer, a desktop computer, and/or the like. In some non-limiting embodiments or aspects, user device 102 may be associated with a care recipient as described herein.

Request processing server 104 may include one or more devices configured to be in communication with user device 102 and/or care provider devices 106 via communication network 108. For example, request processing server 104 may include a server, a group of servers, and/or the like. In some non-limiting embodiments or aspects, request processing server 104 may be associated with a request processer (e.g., an entity that coordinates the provision of services between care providers and care recipients).

Care provider devices 106 include devices configured to be in communication with user device 102, request processing server 104, and/or other care provider devices 106 via communication network 108. For example, care provider devices 106 may include a mobile device (e.g., a smartphone, a tablet, and/or the like), a laptop computer, a desktop computer, and/or the like. In some non-limiting embodiments or aspects, care provider devices 106 may be associated with a care provider as described herein.

Communication network 108 may include one or more wired and/or wireless networks. For example, communication network 108 may include a cellular network (e.g., a long-term evolution (LTE) network, a third generation (3G) network, a fourth generation (4G) network, a code division multiple access (CDMA) network, etc.), a public land mobile network (PLMN), a local area network (LAN), a wide area network (WAN), a metropolitan area network (MAN), a telephone network (e.g., the public switched telephone network (PSTN)), a private network, an ad hoc network, an intranet, the Internet, a fiber optic-based network, a cloud computing network, and/or the like, and/or a combination of some or all of these or other types of networks.

The number and arrangement of systems and/or devices shown in FIG. 1 are provided as an example. There may be additional systems and/or devices, fewer systems and/or devices, different systems and/or devices, or differently arranged systems and/or devices than those shown in FIG. 1. Furthermore, two or more systems and/or devices shown in FIG. 1 may be implemented within a single system or a single device, or a single system or a single device shown in FIG. 1 may be implemented as multiple, distributed systems or devices. Additionally, or alternatively, a set of systems or a set of devices (e.g., one or more systems, one or more devices) of environment 100 may perform one or more functions described as being performed by another set of systems or another set of devices of environment 100.

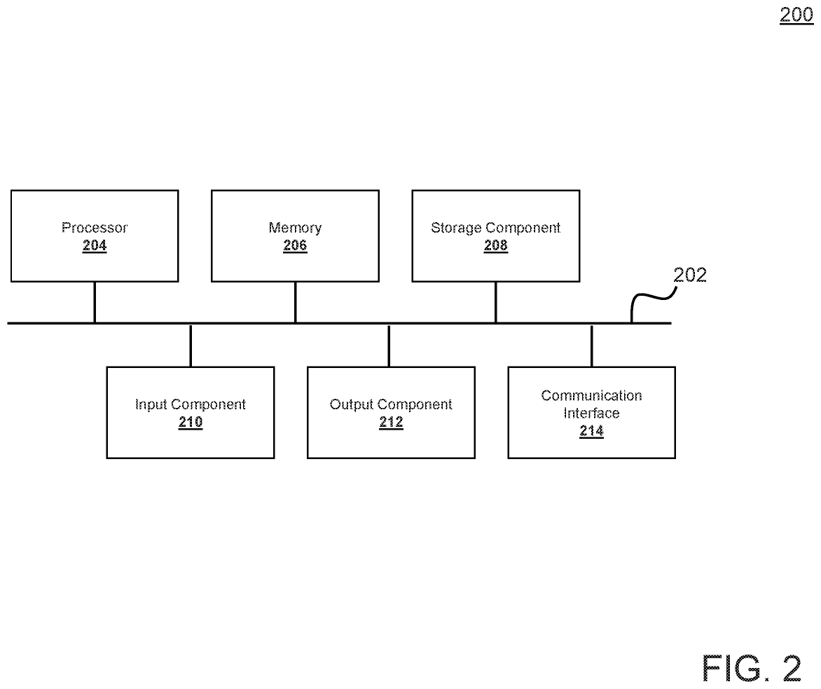

Referring now to FIG. 2, illustrated is a diagram of example components of device 200. Device 200 may correspond to one or more devices of user device 102, one or more devices of request processing server 104, one or more devices of care provider devices 106, and/or one or more devices of communication network 108. In some non-limiting embodiments or aspects, one or more devices of user device 102, one or more devices of request processing server 104, one or more devices of care provider devices 106, and/or one or more devices of communication network 108 may include one or more devices 200 and/or one or more components of device 200. As shown in FIG. 2, device 200 may include bus 202, processor 204, memory 206, storage component 208, input component 210, output component 212, and communication interface 214.

Bus 202 may include a component that permits communication among the components of device 200. In some non-limiting embodiments or aspects, processor 204 may be implemented in hardware, firmware, or a combination of hardware and software. For example, processor 204 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 206 may include random access memory (RAM), read-only memory (ROM), and/or another type of dynamic or static storage device (e.g., flash memory, magnetic memory, optical memory, etc.) that stores information and/or instructions for use by processor 204.

Storage component 208 may store information and/or software related to the operation and use of device 200. For example, storage component 208 may include a hard disk (e.g., a magnetic disk, an optical disk, a magneto-optic disk, a solid state disk, etc.), a compact disc (CD), a digital versatile disc (DVD), a floppy disk, a cartridge, a magnetic tape, and/or another type of computer-readable medium, along with a corresponding drive.

Input component 210 may include a component that permits device 200 to receive information, such as via user input (e.g., a touchscreen display, a keyboard, a keypad, a mouse, a button, a switch, a microphone, a camera, etc.). Additionally or alternatively, input component 210 may include a sensor for sensing information (e.g., a global positioning system (GPS) component, an accelerometer, a gyroscope, an actuator, etc.). Output component 212 may include a component that provides output information from device 200 (e.g., a display, a speaker, one or more light-emitting diodes (LEDs), etc.).

Communication interface 214 may include a transceiver-like component (e.g., a transceiver, a separate receiver and transmitter, etc.) that enables device 200 to communicate with other devices, such as via a wired connection, a wireless connection, or a combination of wired and wireless connections. Communication interface 214 may permit device 200 to receive information from another device and/or provide information to another device. For example, communication interface 214 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 200 may perform one or more processes described herein. Device 200 may perform these processes based on processor 204 executing software instructions stored by a computer-readable medium, such as memory 206 and/or storage component 208. A computer-readable medium (e.g., a non-transitory computer-readable medium) is defined herein as a non-transitory memory device. A memory device includes memory space located inside of a single physical storage device or memory space spread across multiple physical storage devices.

Software instructions may be read into memory 206 and/or storage component 208 from another computer-readable medium or from another device via communication interface 214. When executed, software instructions stored in memory 206 and/or storage component 208 may cause processor 204 to perform one or more processes described herein. Additionally or alternatively, hardwired circuitry may be used in place of or in combination with software instructions to perform one or more processes described herein. Thus, embodiments or aspects described herein are not limited to any specific combination of hardware circuitry and software.

Memory 206 and/or storage component 208 may include data storage or one or more data structures (e.g., a database, and/or the like). Device 200 may be capable of receiving information from, storing information in, communicating information to, or searching information stored in the data storage or one or more data structures in memory 206 and/or storage component 208. For example, the information may include input data, output data, or any combination thereof.

The number and arrangement of components shown in FIG. 2 are provided as an example. In some non-limiting embodiments or aspects, device 200 may include additional components, fewer components, different components, or differently arranged components than those shown in FIG. 2. Additionally or alternatively, a set of components (e.g., one or more components) of device 200 may perform one or more functions described as being performed by another set of components of device 200.

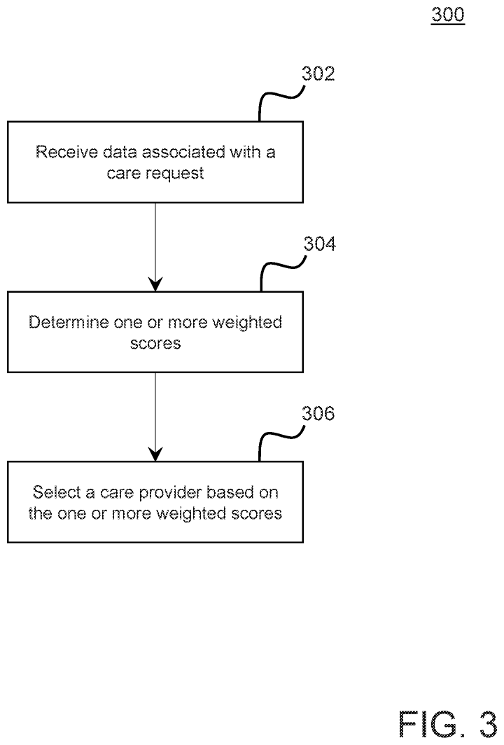

Referring now to FIG. 3, illustrated is a flowchart of a non-limiting aspect or embodiment of a process 300 for determining a care provider based on a care request. In some non-limiting embodiments or aspects, one or more of the functions described with respect to process 300 may be performed (e.g., completely, partially, etc.) by request processing server 104. In some non-limiting embodiments or aspects, one or more of the steps of process 300 may be performed (e.g., completely, partially, and/or the like) by another device or a group of devices separate from and/or including request processing server 104, such as user device 102 and/or one or more care provider devices 106.

As shown in FIG. 3, at step 302, process 300 may include receiving data associated with a care request. For example, request processing server 104 may receive data associated with a care request. In such an example, the care request may represent a request by a care recipient to schedule one or more services with one or more care providers. In some non-limiting embodiments or aspects, request processing server 104 may receive data associated with the care request from user device 102. For example, request processing server 104 may receive data associated with the care request from user device 102 based on user device 102 receiving input. In such an example, user device 102 may receive input from a care recipient, and user device 102 may generate and transmit the data associated with the care request to request processing server 104 based on the input received from the care recipient.

In some non-limiting embodiments or aspects, request processing server 104 may receive data associated with one or more attributes of a care recipient. For example, request processing server 104 may receive data associated with one or more attributes of a care recipient from user device 102. In such an example, request processing server 104 may store the data associated with the one or more attributes of the care recipient in a data structure (e.g., a database) and request processing server 104 may retrieve the data associated with the one or more attributes of the care recipient from the data structure based on (e.g., in response to) receiving data associated with a care request, as described above. Additionally, or alternatively, request processing server 104 may receive the data associated with the one or more attributes of a care recipient from care provider devices 106. For example, request processing server 104 may receive the data associated with the one or more attributes of the care recipient from one or more care provider devices 106 and request processing server 104 may store the data associated with the one or more attributes of the care recipient in a data structure. In such an example, request processing server 104 may retrieve the data associated with the one or more attributes of the care recipient from the data structure based on (e.g., in response to) receiving data associated with a care request, as described above. In some non-limiting embodiments or aspects, the data associated with the one or more attributes of the care recipient may include one or more of data associated with an identifier of the care recipient (e.g., a unique identifier corresponding to the care recipient, a driver's license number corresponding to the care recipient, and/or the like), data associated with a location that is associated with the care recipient (e.g., a home address of the care recipient, a work address of the care recipient, and/or the like), data associated with a date and/or frequency for a care request (e.g., a day, a week, a month, and/or the like), data associated with a requested start time for a care request (e.g., a request to start treatment in the morning or afternoon, a request to start treatment between one or more hours of the day, and/or the like), data associated with a requested end time for a care request (e.g., a request to end treatment by the morning or afternoon, a request to end treatment by one or more hours of the day, and/or the like), an indication of whether the care request is a one-time care request or a repeating care request, data associated with one or more services involved in a care request (e.g., a specification of one or more services to be performed), and data associated with one or more demographic requests that are associated with the care request (e.g., a request that the gender of the care provider match the gender of the care recipient, a request that the age of the care provider be within a certain range of ages, and/or the like). In some non-limiting embodiments or aspects, request processing server 104 may determine one or more values corresponding to the one or more parameters of the care request. For example, request processing server 104 may determine one or more values corresponding to the one or more parameters of the care request based on the data associated with the care request.

In some non-limiting embodiments or aspects, request processing server 104 may receive data associated with one or more rules associated with one or more preferences of a care recipient. For example, request processing server 104 may receive data associated with one or more rules associated with one or more preferences of a care recipient involved in a care request as described herein. In some non-limiting embodiments or aspects, request processing server 104 may receive the data associated with the one or more rules associated with one or more preferences of the care recipient based on request processing server 104 transmitting a request to user device 102. For example, request processing server 104 may receive the data associated with the one or more rules associated with one or more preferences of the care recipient based on request processing server 104 transmitting a request to user device 102, where the request includes a request for input from the care recipient specifying the one or more rules associated with the one or more preferences of the care recipient. In some non-limiting embodiments or aspects, the one or more rules may include, for example, a rule that the gender of a care provider selected matches the gender of the care recipient, a rule that the care provider selected be associated with an address within a predetermined distance of the address associated with the care recipient, and/or the like.

As shown in FIG. 3, at step 304, process 300 may include determining one or more weighted scores. For example, request processing server 104 may determine one or more weighted scores. In some non-limiting embodiments or aspects, request processing server 104 may determine the one or more weighted scores for one or more care providers based on the one or more values corresponding to the one or more parameters of the care request. For example, request processing server 104 may determine the one or more weighted scores for the one or more care providers based on the one or more values corresponding to the one or more parameters of the care request.

In some non-limiting embodiments or aspects, request processing server 104 may compare one or more weighted scores to one or more attributes of one or more care providers. For example, request processing server 104 may compare values of the one or more weighted scores to values of the one or more attributes of the one or more care providers. In such an example, request processing server 104 may determine the one or more weighted scores based on request processing server 104 comparing the values of the one or more weighted scores to the values of the one or more attributes of the one or more care providers. In some non-limiting embodiments or aspects, request processing server 104 may transmit data associated with the one or more weighted scores corresponding to the one or more care providers to user device 102. For example, request processing server 104 may transmit data associated with the one or more weighted scores corresponding to the one or more care providers to user device 102 to enable user device 102 to generate a display including the one or more scores of the one or more care providers. In some non-limiting embodiments or aspects, user device 102 may receive input indicating a selection of a care provider. For example, user device 102 may receive input indicating a selection of a care provider based on user device 102 generating a display including the one or more scores of the one or more care providers. In such an example, user device 102 may transmit data associated with the input to request processing server 104.

In some non-limiting embodiments or aspects, request processing server 104 may select a care provider from among one or more care providers based on one or more rules of a care recipient. For example, request processing server 104 may select a care provider from among one or more care providers based on one or more rules of a care recipient involved in a care request. In such an example, request processing server 104 may select the care provider from among the one or more care providers based on request processing server 104 comparing the one or more rules of the care recipient involved in the care request to the one or more attributes of the care provider in addition to, and/or in the alternative to, the one or more weighted scores of the one or more care providers. In some non-limiting embodiments or aspects, request processing server 104 may select a care provider based on the input received at user device 102. For example, request processing server 104 may select a care provider based on the input received at user device 102 indicating the selection of the care provider.

In some non-limiting embodiments or aspects, request processing server 104 may determine one or more weighted scores based on data associated with a rating of one or more care providers. For example, request processing server 104 may determine one or more weighted scores based on data associated with a rating of one or more care providers that may be involved in a care request. In some non-limiting embodiments or aspects, request processing server 104 may receive the data associated with a rating of one or more care providers from user device 102. For example, user device 102 may receive input from a care recipient associated with user device 102, the input specifying the rating of the one or more care providers. In such an example, user device 102 may transmit data associated with the rating of the one or more care providers to request processing server 104.

In some non-limiting embodiments or aspects, request processing server 104 may receive data associated with the one or more attributes of the one or more care providers. For example, request processing server 104 may receive data associated with the one or more attributes of the one or more care providers based on request processing server 104 determining the one or more weighted scores. In some non-limiting embodiments or aspects, request processing server 104 may receive data associated with the one or more attributes of the one or more care providers, the data associated with the one or more attributes of the one or more care providers including data associated with an identifier of a care provider (e.g., a unique identifier for each care provider), data associated with a location of a care provider (e.g., data associated with a commercial address of a care provider, data associated with a residential address of a care provider, and/or the like), data associated with an availability of a care provider (e.g., one or more days and/or times of day that a care provider is available to provide services), data associated with one or more demographics of a care provider (e.g., an age range of the care provider, a gender of the care provider, and/or the like), data associated with one or more qualifications of a care provider, and/or the like. In some non-limiting embodiments or aspects, the one or more qualifications of the care provider may include a level of experience associated with one or more corresponding care providers, one or more fields (e.g., nursing, primary care physician, cardiologist physician, and/or the like) that one or more care providers have experience in, one or more times (e.g., one or more periods of time such as a month, a year, a plurality of years, and/or the like) associated with an amount of time the one or more care providers have had experience with the one or more fields, one or more certifications attained by a care provider, one or more licenses attained by a care provider, one or more ratings of a care provider, and/or the like. In some non-limiting embodiments or aspects, the data associated with the one or more attributes of the one or more care providers may include data associated with a care provider wellness score representing the mental health and/or wellbeing of one or more care providers.

In some non-limiting embodiments or aspects, request processing server 104 may determine the one or more attributes of the one or more care providers. For example, request processing server 104 may determine the one or more attributes of the one or more care providers based on request processing server 104 transmitting data associated with one or more qualifications to care provider devices 106. In such an example, the data associated with one or more qualifications to care provider devices 106 may include one or more training videos, one or more invitations to communicate with a different care provider (e.g., a coach that assists care providers who are training to provide one or more services) associated with care provider device 106 that is associated with a different care provider, and/or the like. In some non-limiting embodiments or aspects, care provider devices 106 that receive the data associated with one or more qualifications may prompt the care provider operating care provider devices 106 to provide input (e.g., responses to questions, and/or the like) indicating that the care providers are capable of providing one or more services. For example, care provider devices 106 that receive the data associated with one or more qualifications to care provider devices 106 may prompt the care provider operating care provider devices 106 to provide input (e.g., responses to questions associated with the qualifications of the one or more care providers, responses to questions about the mental health and/or wellbeing of one or more care providers, and/or the like) indicating that the care providers are capable of providing one or more services. In such an example, care provider devices 106 that receive the input may transmit data associated with the input provided by the care providers to request processing server 104. In some non-limiting embodiments or aspects, request processing server 104 may determine the one or more attributes of the one or more care providers based on the input provided by the care providers, described above. For example, request processing server 104 may determine the one or more attributes of the one or more care providers based on the input provided by the care providers by request processing server 104 comparing the input provided by the care providers to predetermined inputs associated with correct inputs indicating competency to provide the one or more services.

As shown in FIG. 3, at step 306, process 300 may include selecting a care provider based on the one or more weighted scores. For example, request processing server 104 may select a care provider based on the one or more weighted scores. In some non-limiting embodiments or aspects, request processing server 104 may select the care provider based on the one or more weighted scores, where the selected care provider is associated with a weighted score that is higher than one or more other weighted scores of one or more other care providers. Additionally, or alternatively, request processing server 104 may select the care provider based on the one or more weighted scores, where the selected care provider is associated with a weighted score that is lower than one or more other weighted scores of one or more other care providers.

In some non-limiting embodiments or aspects, request processing server 104 may select a care provider based on an amount of credits (e.g., a type of currency such as funds, credits purchased with funds in advance of the transmission of the data associated with the care request from user device 102, and/or the like). For example, request processing server 104 may select a care provider based on an amount of credits purchased by the care recipient. Additionally, or alternatively, request processing server 104 may select a care provider based on the amount of credits attained by the care recipient where the care recipient is a care provider. For example, request processing server 104 may select a care provider based on the amount of credits attained by the care recipient where the care recipient is a care provider where the amount of credits attained by the care recipient is sufficient to satisfy the amount of credits needed to obtain the services included in the care request from the care provider that was selected. In some non-limiting embodiments or aspects, request processing server 104 may maintain one or more accounts for one or more care recipients and/or one or more care providers. For example, request processing server 104 may maintain one or more accounts for one or more care recipients and/or one or more care providers based on one or more care requests. In such an example, request processing server 104 may maintain one or more accounts for one or more care recipients and/or one or more care providers based on one or more care requests by adding and/or subtracting credits from the one or more accounts for one or more care recipients and/or one or more care providers based on the acceptance and/or completion of services associated with the one or more care requests. In some non-limiting embodiments or aspects, the one or more accounts of the one or more care recipients may be associated with one or more individuals associated with the one or more care recipients (e.g., a parent, a spouse, a child, a guardian, a governmental agency maintaining and/or providing benefits on behalf of the care recipient, a non-profit organization providing benefits on behalf of the care recipient, and/or the like). In some non-limiting embodiments or aspects, an account associated with a care provider may also be associated with a care recipient where the care provider also is involved in one or more care requests as a care recipient.

In some non-limiting embodiments or aspects, request processing server 104 may generate a care request for a care provider. For example, request processing server 104 may generate a care request for a care provider based on request processing server 104 selecting a care provider. In some non-limiting embodiments or aspects, request processing server 104 may generate the care request for the care provider, where the care request includes data associated with the care request received from user device 102. Additionally, or alternatively, request processing server 104 may generate the care request for the care provider, where the care request includes data associated with the care request that is configured to cause a device to display an image representing the care request. For example, request processing server 104 may generate the care request for the care provider, where the care request includes data associated with the care request that is configured to cause care provider device 106 to display an image representing the care request based on (e.g., in response to) care provider device 106 receiving the care request.

In some non-limiting embodiments or aspects, request processing server 104 may transmit data associated with the care request. For example, request processing server 104 may transmit data associated with the care request to care provider device 106. In some non-limiting embodiments or aspects, request processing server 104 may transmit the data associated with the care request to care provider device 106, where care provider device 106 is associated with (e.g., operated by) the care provider that was selected. In some non-limiting embodiments or aspects, care provider device 106 may display an image based on care provider device 106 receiving the data associated with the care request. For example, care provider device 106 may display an image based on care provider device 106 receiving the data associated with the care request, where the image represents the care request.

In some non-limiting embodiments or aspects, the data associated with the care request may include data associated with a personalized message. For example, data associated with the care request may include data associated with a personalized message intended for a care provider that may provide one or more services, where the personalized message specifies one or more of the profile of the care recipient (e.g., a profile indicating whether a care recipient is associated with a dementia diagnosis, a profile indicating whether a care recipient is associated with a post stroke diagnosis, a profile indicating the experience and/or stage in caregiving of the care recipient, a profile including a profile associated with the care provider, a profile indicating a time of day that one or more services are to be provided to the care recipient, a profile indicating one or more days of the week that a service is to be provided, a profile indicating one or more stress areas associated with the care recipient, and/or the like). In some non-limiting embodiments or aspects, request processing server 104 may be configured to perform a process for adapting text based gold standard training associated with one entity (e.g., a government, a country, a state, and/or the like) into another specific international entity associated with environments such as, in a non-limiting example, another country adjusted for cultural, regulatory, and economic differences in a multimedia story based delivery of data associated with a care request. In some non-limiting embodiments or aspects, request processing server 104 may be configured to implement one or more business models for delivering training and/or educational content, subscriptions (e.g., paid-for subscriptions), referrals for care providers, reimbursing family caregivers, and/or the like.

In some non-limiting embodiments or aspects, request processing server 104 may store data associated with a library of media to be presented to one or more care providers. For example, request processing server 104 may store data associated with a library of media to be presented to one or more care providers where the library includes, guides (e.g., guides for providing services for a patient and/or to oneself), tips (e.g., tips for improving physical and/or mental health for a patient and/or oneself), educational material, and/or the like. In some non-limiting embodiments or aspects, request processing server 104 may select one or more instances of media from the library of media. For example, request processing server 104 may select one or more instances of media from the library of media to be provided to a care provider. In such an example, request processing server 104 may select one or more instances of media from the library of media. For example, request processing server 104 may select one or more instances of media from the library of media to be provided to a care provider based on request processing server 104 determining that one or more instances of media are more appropriate than one or more other instances of media (e.g., that the one or more instances of media apply to one or more services involving the care provider, that the one or more instances of media may be useful in improving the physical and/or mental health of the care provider, that the one or more instances of media may be useful in reducing the stress of the care provider, and/or the like. In some non-limiting embodiments or aspects, request processing server 104 may transmit data associated with the one or more instances of media from the library of media to be provided to a care provider. For example, processing server 104 may transmit data associated with the one or more instances of media from the library of media to be provided to a care provider to one or more care provider devices 106.

In some non-limiting embodiments, request processing server 104 may determine an amount of credits associated with a care request. For example, request processing server 104 may determine an amount of credits associated with a care request based on the care request. In some non-limiting embodiments, request processing server 104 may determine an amount of credits associated with a care request based on the care request and a predetermined amount of credits associated with the care request. For example, request processing server 104 may determine an amount of credits associated with a care request based on the care request and a predetermined amount of credits associated with the care request, where the predetermined amount of credits associated with the care request corresponds to a predetermined amount of credits for the one or more services specified by the care request. Additionally, or alternatively, request processing server 104 may determine an amount of credits associated with the care request based on an amount of time spent by a care provider providing services specified by the care request. For example, request processing server 104 may determine an amount of credits associated with the care request based on an amount of time spent by a care provider providing services specified by the care request based on request processing server 104 multiplying a credit rate associated with a care provider and an amount of time spent by the care provider providing the services specified by the care request. In such an example, care provider device 106 associated with the care provider providing the services specified in the care request may transmit data associated with the services provided by the care provider specifying the amount of time spent by the care provider providing the services to request processing server 104. Request processing server 104 may then determine the amount of credits associated with the care request. In some non-limiting embodiments, request processing server 104 may transfer the credits associated with a care request from an account associated with the care recipient involved in the care request to an account associated with the care provider. In some non-limiting embodiments or aspects, request processing server 104 may determine the credit rate associated with a care provider based on the one or more attributes of the one or more care providers, a non-limiting example of which is described above. In some non-limiting embodiments or aspects, credits may be withdrawn from one or more accounts and transferred to one or more personal and/or business accounts.

Further details regarding non-limiting embodiments or aspects of systems, methods, and computer program products for determining a care provider based on a care request are disclosed in Appendix A filed herewith, the entire disclosure of which is hereby incorporated by reference in its entirety.

Although the above methods, systems, and computer program products have been described in detail for the purpose of illustration based on what is currently considered to be the most practical and preferred embodiments or aspects, it is to be understood that such detail is solely for that purpose and that the present disclosure is not limited to the described embodiments or aspects but, on the contrary, is intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect.

What is claimed is:

1. A method, comprising:
receiving data associated with a care request, the data associated with the care request transmitted by a device associated with a care recipient;
determining one or more values corresponding to one or more parameters of the care request;
determining one or more weighted scores corresponding to one or more care providers based on the one or more values corresponding to the one or more parameters of the care request;
selecting a care provider from among the one or more care providers based on the one or more weighted scores;
generating a care request for the care provider selected from among the one or more care providers; and
transmitting the care request to a device associated with the care provider that was selected from among the one or more care providers.

2. The method of claim 1, wherein receiving the data associated with the care request comprises:
receiving data associated with one or more attributes of the care recipient, the one or more attributes of the care recipient comprising:
an identifier of the care recipient,
a rating of the care recipient,
a location associated with the care recipient,
a date for the care request,
the frequency of performance of a service involved in the care request,
a requested start time for the care request,
a requested end time for the care request,
an indication of whether the care request is a one-time care request or a repeating care request,
one or more services involved in the care request, and
one or more demographic requests associated with the care request.

3. The method of claim 1, further comprising:
receiving data associated with or more attributes of a care provider, the one or more attributes of the care provider comprising:
an identifier of the care provider,
a rating of the care provider;
a location associated with the care provider,
availability of the care provider,
clinical skills and competency of the care provider;
one or more times of one or more dates that the care provider is unavailable,
demographics of the care provider,
a method of payment associated with the care request, and
qualifications of the care provider.

4. The method of claim 1, wherein determining the one or more weighted scores corresponding to one or more care providers based on the one or more values corresponding to the one or more parameters of the care request comprises:
determining the one or more weighted scores corresponding to one or more care providers based on comparing values associated with the one or more attributes of the care recipient to values associated with the one or more attributes of the one or more care providers.

5. The method of claim 1, further comprising:
receiving data associated with one or more rules associated with one or more preferences of the care recipient.

6. The method of claim 5, wherein, receiving the data associated with the one or more rules associated with one or more preferences of the care recipient comprises:
transmitting a request to the device associated with the care recipient for input specifying the one or more rules associated with the one or more preferences of the care recipient; and
receiving the data associated with one or more rules associated with one or more preferences of the care recipient,
wherein, selecting the care provider from among the one or more care providers based on the one or more weighted scores comprises:
selecting the care provider from among the one or more care providers based on the one or more weighted scores and one or more rules associated with one or more preferences of the care recipient.

7. The method of claim 1, further comprising:
determining one or more rules associated with one or more preferences of the care recipient based on the one or more attributes of the care recipient,
wherein selecting the care provider from among the one or more care providers based on the one or more weighted scores comprises:
selecting the care provider from among the one or more care providers based on the one or more weighted scores and one or more rules associated with one or more preferences of the care recipient.

8. The method of claim 1, further comprising:
selecting data associated with the one or more instances of media from the library of media based on the one or more care providers, and
transmitting the data associated with the one or more instances of media from the library of media to the device associated with the care provider that was selected from among the one or more care providers.

9. A system, comprising:
at least one processor; and
at least one non-transitory computer-readable medium comprising instructions to direct the at least one processor to perform the method of claim 1.

10. A computer program product, the computer program product comprising at least one non-transitory computer-readable medium including one or more instructions that, when executed by at least one processor, cause the at least one processor to perform the method of claim 1.
